(19) United States
(12) Patent Application Publication
(10) Pub. No.: US 2013/0039833 A1
(43) Pub. Date: Feb. 14, 2013

(54) SYSTEMS AND METHODS FOR PRODUCING AMMONIA FERTILIZER

(71) Applicant: Luca Constantino Zullo, Tonka Bay, MN (US)

(72) Inventor: Luca Constantino Zullo, Tonka Bay, MN (US)

(73) Assignee: LiveFuels, Inc., Menlo Park, CA (US)

(21) Appl. No.: 13/382,882

(22) PCT Filed: Jul. 15, 2010
(86) PCT No.: PCT/US10/42041

§ 371 Date: Feb. 23, 2012

Related U.S. Application Data
(60) Provisional application No. 61/225,853, filed on Jul. 15, 2009.

(51) Int. Cl. C25B 9/00; A01K 61/02; C01C 1/04; C12M 1/00; B01D 61/02

(52) U.S. Cl. 423/359; 435/289.1; 204/242; 210/251; 204/274; 119/200

(57) ABSTRACT

Provided herein are systems and methods for producing ammonia fertilizer for culturing algae and aquaculture. The integrated systems provided herein can be installed on a hydrocarbon production platform and encompasses equipment and processes for generating electricity, preparation of an aqueous electrolyte, hydrogen generation by electrolysis, catalysis of ammonia formation, collection of ammonia, and optionally a nitrogen generator, and a plurality of subsystems for algae culture, and aquaculture.

This application claims the benefit of U.S. Provisional Application No. 61/225,853, filed Jul. 15, 2009, which is incorporated by reference in its entirety.

### 1. INTRODUCTION

Provided herein are systems and methods for producing ammonia fertilizer offshore for the culture of algae, fish, and/or shellfish.

### 2. BACKGROUND OF THE INVENTION

In 2007, a nascent biodiesel industry produced 250 million gallons of a bio-derived diesel substitute from mostly soybean oil in the U.S. However, a practical and affordable feedstock for use in biodiesel has yet to be developed that would allow significant displacement of petrodiesel. It has been proposed to use algae as a feedstock for producing biofuel, including biodiesel. Some algae strains can produce up to 50% of their dried body weight in triglyceride oils. The oil production per acre can be nearly 40 times that of a terrestrial crop, such as soybeans. Moreover, algae do not need arable land, and can be grown in seawater, neither of which competes with terrestrial food crops.

However, culturing algae to produce biofuel requires steady and economical supplies of carbon (C), nitrogen (N), and phosphorous (P). Given C in the form of carbon dioxide can be obtained from the atmosphere, the costs of supplying large amounts of N and P to an algae culture can be a significant expense, particularly where the culture is situated in a remote location or offshore. Although the development of algae presents a feasible option for biofuel production, there is a need to reduce the cost of producing the biofuel from algae. The fall in oil price in late 2008 places an even greater pressure on the fledgling biofuel industry to develop inexpensive and efficient processes. Provided herein are sustainable and economical supplies of nitrogen to an algal culture.

### 3. SUMMARY OF THE INVENTION

Provided herein are integrated systems for producing ammonia fertilizer that can be deployed offshore, or at or near where algae is cultured. In preferred embodiments, the systems are installed on offshore installations originally built for oil and gas production. In certain embodiments, the integrated systems generate hydrogen by electrolysis of water and combine the hydrogen with atmospheric nitrogen to synthesize ammonia. In certain embodiments, the systems provided herein comprise an electrolyzer, an apparatus for generating electric power sufficient for electrolysis, and a reactor for ammonia synthesis. In certain embodiments, the preferred methods for generating electricity is the use of wind power. In some embodiments, the systems provided herein can in addition comprise an apparatus for generating nitrogen from air, an apparatus for desalinating salt water, an apparatus for separating ammonia from unreacted gases, storage for the synthesized ammonia and ammonia fertilizer, and subsystems for handling and storing by-products of the processes provided herein, such as brine from desalination, and oxygen from electrolysis and nitrogen generation. Also encompassed in certain embodiments provided herein are gas compressors, pressure reductors, heat exchangers, an assembly of fluidic connections and equipment that integrate a system provided herein. In certain embodiments, a further feature of the integrated systems is a distribution system of ammonia fertilizer that comprises pipelines and/or risers that are fluidically connected with a plurality of other hydrocarbon production installations, including platforms and subsea structures that are horizontally spaced in a geographic area. In some embodiments, many of these systems are operably associated with parts of a hydrocarbon production installation and can comprise parts of the hydrocarbon production installation repurposed for use in the embodiments provided herein, including decks, subsea structures and pipelines. It is contemplated that the footprint of an integrated system provided herein would allow installation onto hydrocarbon production platforms that are being decommissioned.

In certain embodiments, provided herein are processes for producing ammonia fertilizer comprising generating electric power sufficient for electrolysis, generating hydrogen by electrolysis of an aqueous electrolyte, combining the hydrogen with atmospheric nitrogen to form a synthesis gas, reacting the synthesis gas in the presence of a catalyst and under effective conditions to form ammonia, and collecting the ammonia to form an ammonia fertilizer. In some embodiments, the processes can further comprise prior to electrolysis, preparing the aqueous electrolyte by filtering and/or by desalinating water obtained from an environmental source. In some embodiments, the processes can further comprise generating nitrogen from air by techniques known in the art, such as membrane separation, pressure swing adsorption and cryogenic separation. In some embodiments, the processes can also comprise providing a hydrocarbon production installation on which one or more of the steps of filtering and desalinating water, hydrogen generation by electrolysis, nitrogen generation, ammonia synthesis, and ammonia collection are performed.

In some embodiments, subsystems for maintaining an algae culture and/or an aquaculture are also encompassed. In some embodiments, the algae culture subsystems and the aquaculture subsystems comprise one or more enclosures, and operating subsystems, which include but are not limited to an environmental monitoring subsystem, a feeding subsystem, an aeration subsystem, a transfer mechanism, and a fish processing facility.

### 4. BRIEF DESCRIPTION OF THE DRAWING

FIG. 1 is a process flow diagram of an offshore integrated ammonia production system provided herein.

### 5. DETAILED DESCRIPTION OF THE INVENTION

In certain embodiments, provided herein are sustainable and economical supplies of ammonia fertilizer for fertilizing an aqueous medium used in culturing algae. In certain embodiments, an integrated system for synthesizing ammonia is provided herein, which offers the possibility of fertilizing an area of an ocean for producing algae and aquaculture products, as well as generating credits within a carbon capture and storage regime. Although the embodiments provided herein are described mostly in the context of an offshore installation, it is not intended to be limiting. It is contemplated that the integrated systems and processes provided herein can also be installed in other environments, including coastal, lakeside, and terrestrial locations, and particularly where algae culture and/or aquaculture operations are situated.

In one embodiment, provided herein are integrated systems for synthesizing ammonia (NH₃) characterized by the process by which the two raw materials, hydrogen (H₂) and nitrogen (N₂), are derived from a renewable source obtained locally, and the process which is powered economically by a sustainable energy source. In certain embodiments, an integrated system provided herein comprises an electrolyzer, an apparatus for generating electricity for electrolysis, and a reactor for ammonia synthesis. In some embodiments, the system can further comprise an apparatus for generating nitrogen from air. In some embodiments, the preferred methods for generating electricity is use of wind power. In another embodiment, provided herein is a process for synthesizing ammonia comprising providing an electric current for electrolysis, decomposing water by electrolysis to produce hydrogen, combining the hydrogen with nitrogen obtained from the atmosphere in a reactor under conditions effective to form ammonia, and separating the ammonia for producing ammonia fertilizer or utilization directly as a fertilizer. The term ammonia fertilizer encompasses anhydrous ammonia, hydrous ammonia or ammonium hydroxide at various concentration ranging from about 1% to about 99%, such as about 2%, 5%, 10%, 20%, 30%, 40%, 50%, 60%, 70%, 80%, 90%, and 95%.

Several advantages of the systems provided herein are apparent when they are compared to conventional solutions available for providing nitrogen to fertilize offshore and oceanic areas. First, little or no non-renewable source of energy and raw materials are used in the processes provided herein. The supply of water, which is used as a source of hydrogen, is non-limiting in locations where the systems provided herein are situated. This dramatically reduces the costs associated with acquiring and transporting the raw material. Conventionally, hydrogen for ammonia manufacturing is typically produced from hydrocarbon fuels, such as natural gas, propane, petroleum gas, via chemical reforming using combinations of steam reforming and partial oxidation. Second, electricity for the energy-intensive electrolytic step is generated within the system without relying on the utility grid or fossil fuel. Third, the by-products of the process do not pollute the aquatic environment and can be re-used within the system. For example, heat produced by the exothermic reaction of nitrogen and hydrogen can be used to heat the unreacted gases, or ambient water for algae culture and aquaculture. Oxygen is a by-product of the electrolysis of water and nitrogen generation from air, and can be used for oxygenating the algae culture medium or aquaculture medium. Furthermore, the transportation of conventional N fertilizers, such as urea, ammonium sulfate and ammonium nitrate, to an offshore or oceanic location can add to the cost of growing algae and aquaculture. With the present systems and methods provided herein, there is no need to transport the ammonia fertilizer from site of manufacture to point of application. Overall, the carbon footprint of the processes provided herein is minimal when compared with conventional systems. When the production system is combined with an algae culture, it is possible to accrue credits in a carbon capture and storage regime.

In certain embodiments, the integrated systems provided herein are built by adapting a hydrocarbon production installation for use in the processes provided herein. There are more than 6,500 offshore oil and gas installations around the world, in some 53 countries. The main areas with such installations are Gulf of Mexico: 4,000, Asia: 950, Middle East: 700, North Sea and North East Atlantic: 490, West Africa coast: 380, and South America: 30. The Gulf of Mexico is the most extensively developed and mature offshore petroleum-producing region in the world. In the federally regulated outer continental shelf of the Gulf of Mexico, 40,000 wells have been drilled and nearly 33,000 miles of pipeline are currently in use. When the time arrives that the cost to operate an offshore structure exceeds the income from the hydrocarbons under production, the structure exists as a liability and becomes a candidate for divestiture or decommissioning. Based on data between 1998-2003, the average cost to remove a structure was estimated to be $1,000 per ton. For example, it has been estimated that the cost to remove to shore an 8-pile, 4-well, fixed platform in the Central Gulf of Mexico in 210 feet water depth with a total weight of 980 ton and two pipeline connections would be $1.1 million. Thus, the cost of removal to shore can be significant. One aspect of the present embodiments is to exploit the availability of oil and gas installations that are going to be decommissioned. The use of such installations to produce ammonia fertilizer allows them to remain offshore.

Accordingly, in certain embodiments, provided herein is a hydrocarbon production installation or a part thereof that comprises an integrated system for synthesizing ammonia. The term “hydrocarbon production installation” refers generally to any structure that is installed offshore for the production and processing of oil and/or gas, which includes but is not limited to, various types of drilling platforms and production platforms, as well as pipelines that connect the platforms to each other, to other facilities onshore and on the seabed. The term “platform” generally encompasses hydrocarbon production installation that has a superstructure situated above water and a submerged substructure that provides support, anchorage and/or vertical mooring. In certain embodiments, a superstructure, also referred to as the topside, comprises a plurality of decks and a plurality of modules which house drilling equipment, production equipment, safety equipment, transportation equipment, power generators, and living quarters with catering facilities. In certain embodiments, many elements of the integrated system for synthesizing ammonia are operably associated with the existing structures of the hydrocarbon production installation. By “operably associated” is meant that, continuously or periodically, the system is mechanically, electrically, and/or fluidically connected to parts of a hydrocarbon production installation. In a preferred embodiment, the deck comprises one or more wind turbines to provide electricity for the platform and electrolysis.

In certain embodiments, a platform provided herein comprises an integrated system for synthesizing ammonia and producing ammonia fertilizer as described above and optionally a variety of systems for algae culturing and aquaculture. In certain embodiments, in addition to a hydrocarbon production installation-based system for synthesizing ammonia, provided herein is a body of water containing algae proximate to the hydrocarbon production installation, and a plurality of aquatic organisms that feed on algae populating the body of water. In some embodiments, certain species of algae and/or aquatic organisms are confined in enclosures. Non-limiting examples of an enclosure include a cage, a porous container, a substrate to which shellfishes are attached, a fluidic barrier, and a compartment of a hydrocarbon production installation.

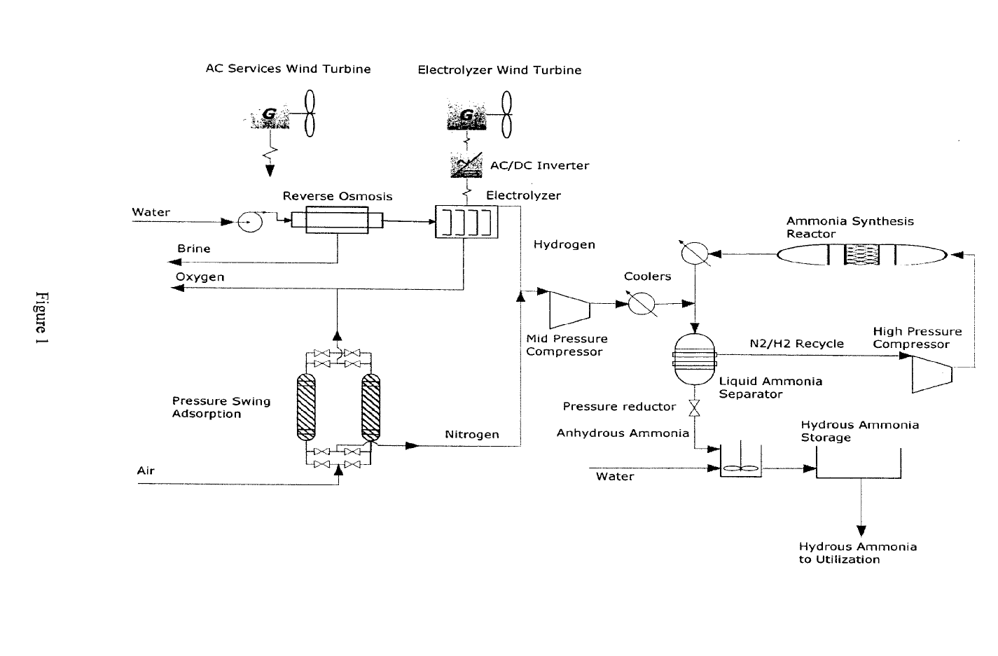

For clarity of disclosure, and not by way of limitation, the detailed description of the integrated systems and processes provided herein is divided into the paragraphs which follow. FIG. 1 is a flow diagram of an exemplary integrated system provided herein and the mass balance and energy demand of such an exemplary system is provided in section 6. As used herein, “a” or “an” means at least one, unless clearly indicated otherwise. The term “about,” as used herein, unless otherwise indicated, refers to a value that is no more than 20% above or below the value being modified by the term. The term “proximate to” is used herein to describe, in context, a functionally effective distance relative to a location, such as a platform provided herein. Without limitation, the term “proximate to” means a distance of about 1 to 10 meters, about 10 to 5000 meters, or about 10, 20, 25, 50, 75, 100, 200, 250, 500, 750, 1000, 2000, 2500, or 5000 meters, or any intermediate distances.

In certain embodiments, the systems provided herein comprise an apparatus for generating electricity to provide power to various equipment and processes, including the electrolysis of water. In certain embodiments, the apparatus for generating electricity includes but is not limited to systems for capturing and converting kinetic energy in or associated with the natural flow of fluids in an environment into electricity, such as but not limited to wind power, hydropower, tidal power, wave power, and/or power by undersea currents. In some embodiments, also contemplated is the use of solar power and geothermal power. Systems for harnessing energy from such sources are well known in the art and are encompassed in the systems provided herein. In a preferred embodiment, wind power is used to generate electricity for the systems and processes provided herein. In some embodiments, such a system comprises a wind turbine that is coupled to a generator for electricity. In some embodiments, one or more wind turbines can be installed on a platform to produce electricity. Many such wind power generating apparatus arc known and can be adopted for use on the platform, see, for example U.S. Pat. No. 4,979,871, U.S. Pat. No. 7,126,235, U.S. Pat. No. 7,347,667 and U.S. Pat. No. 7,329,099. As certain embodiments involve electrolysis in which a direct current is preferred, the systems provided herein can comprise a power conditioner, such as an AC/DC inverter. In some embodiments, the systems provided herein can be designed to operate only when sufficient wind power is available to synthesize ammonia, and comprise a battery system for storing surplus power and provide power during startup. In other embodiments, a diesel generator, preferably using biodiesel, can be employed at startup.

The processes provided herein produces hydrogen by decomposing water molecules (H₂O) in an aqueous electrolyte into oxygen gas (O₂) and hydrogen gas with an electric current. In certain embodiments, the aqueous electrolyte is water that can be obtained from a continuous freshwater source, such as a lake, a stream, or a spring. In other embodiments, the aqueous electrolyte can be desalinated water which can be obtained by desalination of salt water, such as brackish water or seawater. In some embodiments, a continuous source of salt water includes but is not limited to seawater, brackish water, and salt water from an inland salt lake. In some embodiments, a preferred source is the body of seawater that is proximate to an offshore hydrocarbon production installation. Any desalination methods and equipment known in the art can be used, including but not limited to reverse osmosis and/or evaporation. In one embodiment, a process provided herein comprises desalinating salt water by reverse osmosis to prepare an aqueous electrolyte. In a preferred embodiment, the reverse osmosis process, such as the compression of the salt water, is powered electrically by the system. In another embodiment, a process provided herein comprises using one or more multiple effect evaporators to remove the salt in salt water to prepare an aqueous electrolyte. In a preferred embodiment, the evaporation process is effected by heat produced by the exothermic reaction of ammonia synthesis. In certain embodiments, water is withdrawn from its source by a pump (pressure ranging from 2 to 4 bars), and passed through a filtering apparatus, i.e., one or more filters, to remove suspended particles, planktons, and colloids. In some embodiments where more than one filter is used in sequence, the porosity of the filters progressively decrease from about 100 microns to 20 microns, and then to 2 microns. Accordingly, in certain embodiments, the systems provided herein comprise a pump for the intake of water, a filtering apparatus to remove particulate matters, and optionally a desalinating apparatus to remove salt, such as one or more reverse osmosis cells or multiple effect evaporators. In certain embodiments, a system provided herein further comprises an assembly of pipes to conduct the water withdrawn from a source to the filtering apparatus and/or desalinating apparatus, and the aqueous electrolyte from the filtering apparatus and/or desalinating apparatus to the electrolyzer, and valves, sensors, and controls to maintain the operating parameters of the processes.

In certain embodiments, the systems provided herein further comprise an electrolyzer, which comprise an aqueous electrolyte disposed between and in ionic communication with electrodes, wherein the electrodes are electrically connected to a source of electric current. In certain embodiments, the electrodes can be separated by membranes in individual chambers. In some embodiments, depending on the desired rate of hydrogen production, more than one electrolyzer can be used. Electrolysis is performed by application of an electric current to the electrodes, and oxygen is produced at the anode, while hydrogen is produced at the cathode. In a preferred embodiment, the electric current is a direct current generated by a wind turbine of the system. In one embodiment, the electrolyzer further comprises apparatus for separating gases produced during electrolysis, including but not limited to phase separators, such as a hydrogen gas separator and/or an oxygen gas separator, which are respectively fluidically connected to a hydrogen outlet and an oxygen outlet. In one embodiment, the hydrogen outlet provides a hydrogen stream for mixing with nitrogen for the synthesis reaction, or is connected to a hydrogen storage. In one embodiment, the oxygen outlet is connected to an oxygen storage, a subsystem for delivering oxygen to an algae culture and/or aquaculture, or a vent for releasing the gas into the atmosphere. In one embodiment, as water is consumed by the process, the aqueous electrolyte is provided to the electrolyzer in a controlled manner through an electrolyte inlet. Many commercially available electrolyzers can be adapted for use in the systems provided herein by one skilled in the art without undue experimentation, see for example, U.S. Pat. No. 6,033,549 and U.S. Pat. No. 6,652,719.

In certain embodiments, the processes provided herein synthesize ammonia according to the Haber-Bosch process and generally comprise mixing a hydrogen stream with a nitrogen stream to form a synthesis gas stream, passing the unreacted synthesis gas stream over an ammonia catalyst under pressure to synthesize ammonia, and collecting the ammonia from the reacted synthesis gas stream. In certain embodiments, hydrogen gas and nitrogen gas are blended into a single stream of synthesis gas and fed into a compressor that brings the stream to about 25 to 120 atms, preferably about 50 atms. In a preferred embodiment, the synthesis gas comprises hydrogen and nitrogen in a molar ratio of about 3 to 1. In one embodiment, the unreacted gas stream is compressed to about 200 atms and can be mixed with recycling reacted synthesis gas before entering the reactor. In one embodiment, any reactor comprising one or more ammonia catalysts known in the art can be used and conversion in excess of 90% can be obtained. The reaction is exothermic, releasing about 5411.7 kJ/kg of NH₃. In one embodiment, the reacted synthesis gas can be cooled to below the dew point of ammonia by indirect heat exchange with colder fluids, such as an unreacted synthesis gas stream, or ambient water. The cooling reduces the pressure and allows the ammonia to condense into liquid form. In one embodiment, the heat released by the exothermic synthesis reaction can be used for a variety of purposes, including but not limited to, desalination of salt water by evaporation, or maintaining culture medium or a body of water proximate to the system at a temperature optimal for algae growth and/or aquaculture. In one embodiment, condensed liquid ammonia is separated from the reacted synthesis gas and stored as anhydrous ammonia or as hydrous ammonia after mixing with water. In a preferred embodiment, a system provided herein comprises an ammonia recycle loop wherein reacted synthesis gas is recycled by mixing with freshly prepared unreacted synthesis gas. In one embodiment, the recycling stream is typically 10 to 15 times the stream of fresh synthesis gas. In one embodiment, the system provided herein can comprise, in addition to a reactor comprising ammonia catalyst for ammonia synthesis, a stream of unreacted synthesis gas, a recycling stream of synthesis gas, a compressor for conditioning the synthesis gas for the catalytic synthesis of ammonia, an assembly of pipes for conducting the unreacted synthesis gas stream, the recycling synthesis gas stream, the reacted synthesis gas stream, and ammonia within the system, an ammonia recycle loop, heat exchangers, pressure reductors, phase separators, and valves, sensors, and controls for maintaining desired operating parameters of the synthesis reaction.

In certain embodiments, the processes provided herein can further comprise producing a nitrogen stream by generating nitrogen from atmospheric air, or by purging oxygen from the atmospheric air intake or from the recycling synthesis gas stream. In certain embodiments, any method known in the art for generating nitrogen can be used, including but not limited to, pressure swing adsorption, selectively permeable membrane separation, or cryogenic rectification. In one embodiment, a by-product of producing a nitrogen stream is oxygen gas which can be vented to the atmosphere, stored, or utilized in oxygenating culture medium for algae culture and/or aquaculture. Accordingly, in certain embodiments, the systems provided herein can further comprise a pressure swing adsorption-type nitrogen generator, a permeable membrane separator, or a cryogenic rectification separator, and an assembly of pipes, filters, and valves for regulating the intake of air and conducting a nitrogen stream, and optionally an oxygen stream.

In certain embodiments, the integrated systems provided herein can further comprise chambers for mixing anhydrous ammonia with water, storage for hydrous ammonia, and a distribution system of ammonia fertilizer to points of application, to other storage facilities, or to a vessel for transportation. In some embodiments, systems for distributing hydrous ammonia as a fertilizer can include a network of pipelines that connect hydrocarbon production installations with one another, with vessels, or with shore facilities; and risers that fluidically connect the seabed to the surface or water at any intermediate depth. In some embodiments, hydrocarbon production installations such as pipelines, risers, manifolds, pumps, and the likes, can be used to transport ammonia, ammonia fertilizer, culture medium enriched with ammonia, or culture medium containing algae. In some embodiments, culture medium comprising ammonia and/or algae can be transported in the network to various locations within a geographic area. One aspect provided herein comprises transporting a stream of culture medium or water that has a higher concentration of ammonia fertilizer to a body of oligotrophic water, optionally admixing the stream with the body of oligotrophic water. In some embodiments, the methods provided herein also comprise transporting and discharging ammonia, ammonia fertilizer, culture medium enriched with ammonia, or culture medium containing algae into an enclosure or a body of water proximate to the system.

In certain embodiments, in addition to the above equipment, the integrated system can further comprise various other equipment well known in the chemical industry and the hydrocarbon production industry, such as valves (e.g., relief valves, check valves, manual valves, actuated valves, needle valves, and the like), filters (e.g., deionization bed cartridge(s), filter cartridge(s), and the like), sensors (e.g., pressure, temperature, flow, humidity, conductivity, gas mixture, water level, and the like), controls including but not limited to temperature controllers (such as, heaters, heat exchangers, coolers, dryers, and the like), pressure controllers (such as, compressors, and the like), flow controllers (such as, pumps, fans, blowers, and the like), conduits (e.g., fluid conduits, electrical conduits, and the like). It should be noted that redundant system components or merely additional components can be employed, in parallel or series operation.

In certain embodiments, subsystems for culturing algae and/or aquaculture subsystems are installed alongside or proximate to an integrated system provided herein. In certain embodiments, an algae culture subsystem can comprise equipment for sampling water, modules for monitoring algal growth and species diversity, facilities to grow algae in a body of water proximate to the platform, apparatus for concentrating and distributing algae, and a network of pipes for transporting algae. In some embodiments, an aquaculture subsystem is also installed on or near a platform which comprises an assemblage of fishes and/or shellfishes that are active at multiple trophic levels. In some embodiments, the aquaculture subsystem can comprise hatchery facilities for hatching and rearing fishes and shellfishes, apparatus for feeding the fishes/shellfishes, instruments for monitoring the aquatic environment for fish/shellfish culture, apparatus for aeration of water at various depths, apparatus for transferring fishes/shellfishes, apparatus for harvesting fishes/shellfishes, and facilities for processing fish oil and fish meal, and rendering shellfishes. In some embodiments, the culturing of fishes and shellfishes in the body of water can entail replenishing a body of water proximate to the platform with algae; and/or maintaining the level of dissolved oxygen in the water favorable for growth of fishes and shellfishes.

In certain embodiments, it is contemplated that the systems and methods provided herein can be used in many of the coastal waters in the United States and worldwide where hydrocarbon production installations are installed. The terms “ocean” and “sea” are used interchangeably, and are taken to represent the bodies of salt water which cover the surface of the earth. The term “coast” and “offshore” are used interchangeably to include all areas between land and ocean, such as but not limited to, estuaries and coastal ocean. A coast can be classified as open continental shelf (e.g., Georgia Bight, Monterey Bay, Louisiana Shelf), coastal embayment (e.g., Massachusetts Bay, Buzzards Bay, Long Island Sound), river plume estuary (e.g., Mississippi River Plume), coastal plain or drowned river valley estuary (e.g., Chesapeake Bay, Hudson River, Charleston Harbor, Choctawhatchee Bay, Perdido Bay, Apalachee Bay), coastal plain salt marsh estuary (e.g., Plum Island Sound, North Inlet, Duplin River, Pensacola Bay), lagoon (e.g., Padre Island, Pamlico Sound, Apalachicola Bay), fjord estuary (e.g., Penobscot Bay), coral reef system (e.g., Kaneohe Bay), tectonically-caused estuary (e.g., San Francisco Bay, Tomales Bay), large river with non-drowned river estuary (e.g., Columbia River), seagrass-dominated estuary (e.g., Tampa Bay, lower Perdido Bay), rocky-intertidal macroalgae dominated estuary (e.g., Casco Bay).

Technical and scientific terms used herein have the meanings commonly understood by one of ordinary skill in the art to which the present embodiments pertain, unless otherwise defined. Reference is made herein to various methodologies known to those of skill in the art. Publications and other materials setting forth such known methodologies to which reference is made are incorporated herein by reference in their entireties as though set forth in full. The practice of the embodiments provided herein will employ, unless otherwise indicated, techniques of chemical engineering, and the algae culture and aquaculture industries, which are within the skill of the art.

### 6. PREFERRED EMBODIMENTS

By the features of the system and processes described above, it is possible to provide a system capable of synthesizing ammonia in an offshore location, with minimal provision of raw material and energy derived from fossil fuel. Referring to FIG. 1, an integrated system and processes provided herein are generally illustrated. Assuming that a 1 MW electrolyzer is used in the system, the following are estimations of mass balance for processes provided herein.

Hydrogen production: given 1 MWh of wind power for an electrolyzer, and assuming that the efficiency of an AC/DC inverter is 95%, and the efficiency of the electrolyzer is 65%, the stoichiometric power demand of water electrolysis is about 5.6 kWh/kg H_{2.} The rate of hydrogen production is at about 110.3 kg/h and the demand for fresh water as an aqueous electrolyte is about 1527 kg/h.

Ammonia production: given the theoretical stoichiometric yield is 5.67 kg NH₃/kg H₂, at 90% efficiency of catalytic conversion, the yield is at about 5.1 kg NH₃/kg H₂ The total hourly production is about 562.5 kg/h of ammonia (about 463.2 kg/hr of N). The capacity factor of the turbine is 35% and yearly ammonia production is about 1,725 ton/yr (about 1,420 ton/yr of N).

Nitrogen demand: given the theoretical stoichiometric yield 1.21 kg NH₃/kg N₂ at 90% efficiency of catalytic conversion, the yield is about 1.08 kg NH₃/kg N_{2.} Nitrogen demand is estimated at about 520.8 kg/h. Assuming pressure swing adsorption is used to generate nitrogen at an efficiency of 90%, the demand of air is about 526.58 sem/h.

Besides the energy used by the electrolyzer, the other major power consumption occurs in the compression of synthesis gas in the ammonia recycle loop. An estimated typical demand for power in the ammonia recycle loop is about 10 GJ/ton NH₃. The base case demand is estimated at 5.62 GJ/h or 1.55 MWh. Assuming that 1 MWh is required for other services, such as pumps, fans, compression for pressure swing adsorption, etc, the total service demand is estimated to be about 2.55 MWh, and the total demand at about 3.55 MWh. An installation of power generator to supply 4.00 MW is preferred.

In certain embodiments, an overall 22.7 GJ of renewable energy is used in the integrated system to produce a ton of ammonia. In contrast, it is estimated that greater than 30 GJ of non renewable resources (such as, natural gas feedstock and fossil fuel-generated power) are required to produce a ton of ammonia by conventional methods. Therefore, by integration of the processes of electrolysis and ammonia synthesis, and configuring the system on a hydrocarbon producing installation where water and wind power is abundant, an impressive and unexpected saving of energy and reduced use of non-renewable resources are achieved. Furthermore, the systems provided herein can advantageously support the growth of algae and aquaculture products, such as fish and shellfish.

Algae culture: it is estimated that 1 ton of reactive nitrogen supports the primary fixation 6.25 tons of carbon in phytoplankton, and assuming that other nutrients are not limited and 100% uptake of nitrogen into phytoplankton, a 1,420 t/yr of nitrogen leads to the uptake of 8,875 ton/yr of dissolved inorganic carbon, that is equivalent to 32,500 ton/yr of CO2. Assuming that on average 44% of the dry weight of phytoplankton is carbon, 1420 t/yr of nitrogen leads to the production of 20,170 ton/yr (db) of algae.

Aquaculture: taking into consideration the efficiency in trophic level transfers, i.e. 1 ton of reactive nitrogen supports approximately 10 tons of wet foraging fish weight, 1420 t/yr of nitrogen leads to the production of 14,200 t/yr (31.2 million lb/yr) of fish.

All references cited herein are incorporated herein by reference in their entirety and for all purposes to the same extent as if each individual publication or patent or patent application was specifically and individually indicated to be incorporated by reference in its entirety for all purposes.

Many modifications and variations of the embodiments provided herein can be made without departing from its spirit and scope, as will be apparent to those skilled in the art. The specific embodiments described herein are offered by way of example only, and the embodiments provided herein are to be limited only by the terms of the appended claims along with the full scope of equivalents to which such claims are entitled.

What is claimed:

1. An integrated system for producing ammonia fertilizer for algae culture or aquaculture, wherein said system comprises an electrolyzer, an apparatus for generating electric power sufficient for electrolysis of an aqueous electrolyte, and a reactor comprising a catalyst for ammonia synthesis.

2. The system of claim 1, wherein said apparatus for generating electric power converts kinetic energy in the natural flow of fluids in an environment into electricity.

3. The system of claim 1, wherein said apparatus for generating electric power comprises a wind turbine and an AC/DC inverter.

4. The system of claim 1, wherein said system further comprises an apparatus for generating nitrogen from air.

5. The system of claim 4, wherein said apparatus for generating nitrogen comprises a pressure swing adsorption-type nitrogen generator, a permeable membrane separator, or a cryogenic rectification separator.

6. The system of claim 1, wherein said system further comprises an apparatus for desalinating brackish water, salt water, or seawater.

7. The system of claim 6, wherein said desalinating apparatus comprises at least one reverse osmosis cell, and/or at least one multiple effect evaporator.

8. The system of claim 1, wherein said system further comprises an effluent stream of oxygen produced by electrolysis and/or an apparatus for generating nitrogen from air.

9. The system of claim 1, wherein said system further comprises a stream of unreacted synthesis gas that feeds into a recycling stream of synthesis gas that comprises ammonia, a compressor that is connected to an inlet to the reactor, a heat exchanger and a phase separator that are connected to an outlet from the reactor.

10. The system of claim 1, wherein said system further comprises apparatus for storing ammonia and/or apparatus for mixing ammonia with water to form an ammonia fertilizer.

11. The system of claim 1, wherein said system further comprises a subsystem for culturing algae and/or an aquaculture subsystem.

12. The system of claim 11, where said subsystem for culturing algae comprise a body of water populated with algae that is proximate to the system.

13. The system of claim 11, where said aquaculture subsystem comprise a body of water that is proximate to the system, wherein said body of water is populated with algae and aquatic organisms that feed on the algae.

14. A hydrocarbon production installation-based system for synthesizing ammonia, said system comprising an electrolyzer, an apparatus for generating electric power sufficient for electrolysis of an aqueous electrolyte, and a reactor comprising a catalyst for ammonia synthesis.

15. The system of claim 14, wherein said apparatus for generating electric power converts kinetic energy in the natural flow of fluids in an environment into electricity.

16. The system of claim 14, wherein said apparatus for generating electric power comprises a wind turbine and an AC/DC inverter.

17. The system of claim 14, wherein said system further comprises an apparatus for generating nitrogen from air.

18. The system of claim 17, wherein said apparatus for generating nitrogen comprises a pressure swing adsorption-type nitrogen generator, a permeable membrane separator, or a cryogenic rectification separator.

19. The system of claim 14, wherein said system further comprises an apparatus for desalinating brackish water, salt water, or seawater.

20. The system of claim 19, wherein said desalinating apparatus comprises at least one reverse osmosis cell, and/or at least one multiple effect evaporator.

21. The system of claim 14, wherein said system further comprises an effluent stream of oxygen produced by electrolysis and/or an apparatus for generating nitrogen from air.

22. The system of claim 14, wherein said system further comprises a stream of unreacted synthesis gas that feeds into a recycling stream of synthesis gas that comprises ammonia, a compressor that is connected to an inlet to the reactor, a heat exchanger and a phase separator that are connected to an outlet from the reactor.

23. The system of claim 14, wherein said system further comprises apparatus for storing ammonia and/or apparatus for mixing ammonia with water to form an ammonia fertilizer.

24. The system of claim 14, wherein said system further comprises a subsystem for culturing algae and/or an aquaculture subsystem.

25. The system of claim 24, where said subsystem for culturing algae comprises a body of water populated with algae that is proximate to the system.

26. The system of claim 24, where said aquaculture subsystem comprise a body of water that is proximate to the system, wherein said body of water is populated with algae and aquatic organisms that feed on the algae.

27. A process for producing ammonia fertilizer, said process comprising (i) generating electric power sufficient for electrolysis by at least one wind turbine, (ii) generating hydrogen by electrolysis of an aqueous electrolyte, (iii) combining the hydrogen with nitrogen to form a synthesis gas, (iv) reacting the synthesis gas in the presence of a catalyst and under effective conditions to form ammonia, and (v) collecting the ammonia to form an ammonia fertilizer.

28. The process of claim 27, further comprising (vi) preparing the aqueous electrolyte by filtering and/or desalinating water obtained from an environmental source.

29. The process of claim 27, further comprising (vii) generating nitrogen from air by pressure swing adsorption.

30. The process of claim 27, further comprising providing a hydrocarbon production installation on which steps (i) through to (v) is performed.
